(19) United States
(12) Patent Application Publication
(10) Pub. No.: US 2025/0325204 A1
(43) Pub. Date: Oct. 23, 2025

(54) CONTINUOUS ANALYTE METER HAVING FLEXIBLE ELECTROCHEMICAL SENSOR

(71) Applicant: UXN Co., Ltd., Gyeonggi-do (KR)

(72) Inventor: Jun Young PARK, Gyeonggi-do (KR)

(21) Appl. No.: 18/278,042

(22) PCT Filed: May 23, 2023
(86) PCT No.: PCT/KR2023/006985

§ 371 Date: Dec. 13, 2024

(30) Foreign Application Priority Data
May 23, 2022 (KR) 10-2022-0063091

(51) Int. Cl. A61B 5/1473; A61B 5/145

(52) U.S. Cl. CPC A61B 5/1473; A61B 5/14532; A61B 2562/164

(57) ABSTRACT

A continuous analyte meter may include an electrochemical sensor including a distal portion having a plurality of electrodes reacting with an analyte in the body and a proximal portion having a plurality of sensor pads connected to the electrodes, and a transmitter attached to the skin, the transmitter including a main substrate on which at least one of a power supply unit, a communication unit, and a control unit is formed, and a housing in which the main substrate is accommodated.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2023/006985, filed May 23, 2023, which claims priority to the benefit of Korean Patent Application No. 10-2022-0063091 filed in the Korean Intellectual Property Office on May 23, 2022, the entire contents of which are incorporated herein by reference.

### BACKGROUND

### 1. Technical Field

The present disclosure generally relates to a continuous analyte meter using an electrochemical sensor at least partially invasively inserted into the body to continuously measure an analyte.

### 2. Background Art

When an inserter is taken as the reference position, a first end of an electrochemical sensor connected to a main substrate may be referred to as a proximal portion because it is located close to the inserter, and a second end of the electrochemical sensor inserted into the body may be referred to as a distal portion because it is located far from the inserter.

The proximal portion of the electrochemical sensor may be electrically connected to a main substrate of a transmitter, and at least a portion of the distal portion of the electrochemical sensor may be inserted into the body. The proximal portion and the distal portion may be located opposite to each other. The proximal portion of the electrochemical sensor may be electrically connected to the main substrate of the transmitter, which includes an electric circuit required to measure an analyte including glucose.

The transmitter may be placed inside the inserter along with the electrochemical sensor prior to being attached to the skin. A type in which the transmitter and the electrochemical sensor are already combined may be referred to as an all-in-one type transmitter.

In order to relieve pain during invasive insertion and reduce discomfort during wearing, a base layer of the electrochemical sensor can be flexible, and the thickness and size of the electrochemical sensor need to be minimized.

As the size of the electrochemical sensor decreases, the area of an electrode formed at the distal portion may also decrease. When the area of the electrode is not sufficiently secured, signal disturbance may occur due to noise. Thus, during manufacture of the electrochemical sensor, it is required to consider both aspects of reducing the size of the sensor and securing the area of the electrode.

### SUMMARY

An electrochemical sensor is required to have good flexibility, small size, narrow width, and thin thickness in order to relieve pain upon insertion into the body and reduce discomfort upon wearing.

The present disclosure is intended to propose an electrochemical sensor that is flexible and thin enough to be impossible to penetrate the skin alone without a needle, thereby relieving pain and reducing discomfort.

A continuous analyte meter according to the present disclosure may include: an electrochemical sensor including a distal portion having a plurality of electrodes reacting with an analyte in the body and a proximal portion having a plurality of sensor pads connected to the electrodes; and a transmitter attached to the skin, the transmitter including a main substrate on which at least one of a power supply unit, a communication unit, and a control unit is formed, and a housing in which the main substrate is accommodated.

Here, the distal portion of the electrochemical sensor may be disposed at an exposed portion of a needle exposed along a longitudinal direction of the needle, the distal portion of the electrochemical sensor may be inserted into the body after the skin is incised by the needle, the electrochemical sensor may have flexibility that it is impossible to penetrate the skin alone without the needle, and the electrochemical sensor may include a flexible base layer, a conductive layer stacked on the base layer, and an insulating layer attached on the conductive layer.

According to a continuous analyte meter according to the present disclosure, by using a flexible and miniaturized electrochemical sensor, it is possible to relieve pain and reduce discomfort upon invasive insertion of the electrochemical sensor into the body.

According to the continuous analyte meter according to the present disclosure, it is possible to reduce the thickness of the electrochemical sensor by simplifying a manufacturing method of the sensor, to form a large number of electrodes or leads in a narrow width of the sensor by forming a trench with a minimum width by laser etching, and to maximize the width of the electrodes or leads. This can improve electrical insulation between wirings, can be advantageous in blocking signal noise, and enables accurate data to be secured.

According to the continuous analyte meter according to the present disclosure, it is possible to eliminate generation of burrs on a cut surface of a metal conductive layer by precise processing such as laser etching, to eliminate generation of foreign substances between conductive islands, and to ensure insulation even with a minimized width of the trench, thereby guaranteeing the degree of freedom in wiring design. In addition, it is possible to secure sufficient electrode and sensor pad areas, thereby improving measurement accuracy and reducing a software burden required for signal processing.

### BRIEF DESCRIPTION OF THE DRAWINGS

FIG. 1 is a sectional side view illustrating an inserter and a transmitter according to the present disclosure.

FIG. 2 is an assembled perspective view illustrating an electrochemical sensor and a needle according to an embodiment of the present disclosure.

FIG. 3 is a plan view of FIG. 2.

FIG. 4 is an assembled perspective view illustrating an electrochemical sensor and a needle according to another embodiment of the present disclosure.

FIG. 5 is a plan view of FIG. 4.

FIG. 6 is a view illustrating a comparative embodiment for forming electrodes at a distal portion.

FIG. 7 is a view illustrating a method of manufacturing the electrochemical sensor according to the present disclosure.

FIG. 8 is a view illustrating a conductive island and a trench according to the present disclosure.

FIG. 9A is a side view illustrating the electrochemical sensor according to the present disclosure, and FIG. 9B is a plan view illustrating the electrochemical sensor according to the present disclosure.

FIG. 10 is a plan view illustrating an electrochemical sensor array according to the present disclosure.

### DETAILED DESCRIPTION

Hereinafter, a case in which an electrochemical sensor 400 according to the present disclosure is used in a continuous glucose monitoring system (CGMS) for measuring the concentration of glucose in interstitial fluid or blood will be described as an example. However, a continuous analyte meter according to the present disclosure is not limited to measuring the concentration of glucose in the body and can be extensively applied to continuous analyte meters that measure other bio-markers.

### Inserter

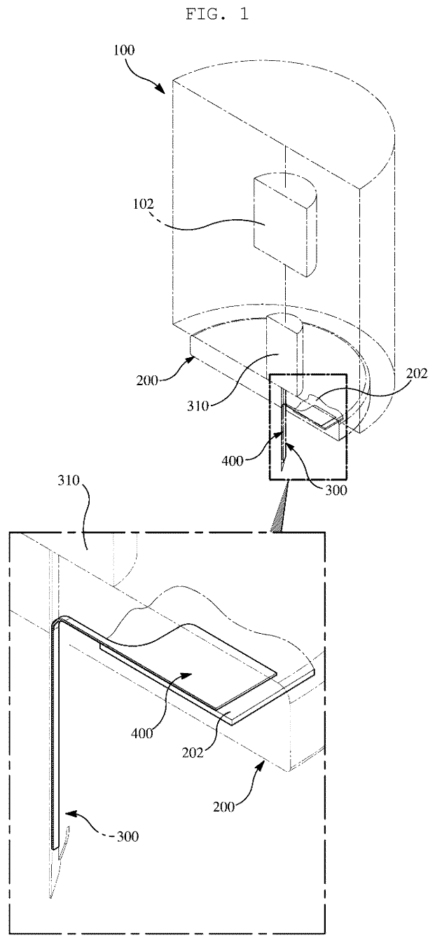

Referring to FIG. 1, the electrochemical sensor 400 according to the present disclosure may be attached to the skin along with a transmitter 200. The transmitter 200 may control a signal measured by the electrochemical sensor 400 and continuously transmit a measured blood glucose level to an external terminal including a mobile phone.

The external terminal may be provided separately from the transmitter 200 attached to the skin, and continuously receive measurement data of the electrochemical sensor 400 wirelessly from the transmitter 200. A user may continuously monitor and diagnose measurement data of the electrochemical sensor 400 for bio-markers including glucose, lactate, and other substances to be measured.

The electrochemical sensor 400 and the transmitter 200 may be provided to the user in a state of being loaded in an inserter 100 before being attaching to the skin. By a user's attachment motion, the electrochemical sensor 400 and the transmitter 200 may be detached from the inserter 100 and attached to the skin.

A first end of the electrochemical sensor 400 connected to an electrical part of the transmitter 200 including a main substrate 202 may be referred to as a proximal portion 402, a second end of the electrochemical sensor 400 at least partially invasively inserted into the body may be referred to as a distal portion 406, and a portion that connects the proximal portion 402 and the distal portion 406 to each other, is disposed between the proximal portion 402 and the distal portion 406, and is flexibly bendable may be referred to as a bending portion 405.

Invasive insertion as used herein may refer to inserting at least a portion of the distal portion 406 of the electrochemical sensor 400 into the body.

The transmitter 200 and the electrochemical sensor 400 may be provided to the user in a state in which they are already connected to each other prior to being attached to the skin.

The transmitter 200 may be located in a first position in a state of being loaded in the inserter 100. The transmitter 200 may be moved from the first position to a second position by a user's motion. The transmitter 200 may be attached to the skin in the second position. An insertion direction of the transmitter 200 and the electrochemical sensor 400 may refer to a direction from the first position to the second position.

A needle 300 may have an exposed portion exposed in the longitudinal direction thereof. A portion of the electrochemical sensor 400 may be disposed inside the needle 300. The needle 300 may serve to incise the skin and guide the electrochemical sensor 400 so that at least a portion of the distal portion 406 is invasively inserted into the body along the insertion direction.

The inserter 100 may include an actuator 102 that operates the transmitter 200 and the electrochemical sensor 400 from the first position to the second position.

The actuator 102 may advance the needle 300 or the transmitter 200 from the first position to the second position so that the needle 300 or the distal portion 406 is inserted into the skin.

After the transmitter 200 and the electrochemical sensor 400 are attached to the skin in the second position, the actuator 102 may retract the needle 300 from the second position to a third position so that the needle 300 is separated from the transmitter 200 and the electrochemical sensor 400.

The actuator 102 may be connected to a needle handle 310 to which the needle 300 is fixed. The needle handle 310 may be detachable from the actuator 102.

An inner space may be provided between an upper cover and a lower cover of the transmitter 200. The main substrate 202 may be seated in the inner space of the transmitter 200.

The main substrate 202 may be provided with at least one of a power supply unit such as a battery required to measure the glucose concentration by the distal portion 406, a control unit including an electric circuit, a wireless communication unit for controlling data measured by the electrochemical sensor 400 and wirelessly transmitting the data to the outside, and an operational amplifier.

The power supply unit may supply a bias voltage that can generate an electrochemical reaction of a working electrode.

A signal of an analyte measured at the distal portion 406 may be amplified by the operational amplifier.

The magnitude of an output current for a given bias on the working electrode may be a measure of the concentration of the analyte, such as glucose, in the vicinity of an electrode 424.

The control unit including the electrical circuit may control the electrical potential between the working electrode and a reference electrode at one or more preset values.

A first surface of the electrochemical sensor 400 on which a sensor pad 428 is formed may face the main substrate 202, and a second surface of the electrochemical sensor 400 may be exposed to the inner space of the transmitter 200.

The sensor pad 428 may be formed at the proximal portion 402 of the electrochemical sensor 400. A contact pad electrically connected to the sensor pad 428 may be formed on the main substrate 202.

Since at least a portion of the electrochemical sensor 400 is invasively inserted into the skin, the electrochemical sensor 400 or a base layer 410 may be flexible to relieve pain during invasive insertion and reduce discomfort during wearing.

The distal portion 406 of the electrochemical sensor 400 may be disposed at the exposed portion of the needle 300 exposed along the longitudinal direction thereof. An end of the needle 300 may be in a more protruding position than an end of the distal portion 406. The distal portion 406 of the electrochemical sensor 400 may be inserted into the body after the skin is incised by the needle 300.

Pain relief and discomfort reduction are key performances of the continuous analyte meter from the user's point of view. To this end, the electrochemical sensor 400 has flexibility that it is impossible to penetrate the skin alone, and the electrochemical sensor 400 is thin and flexible enough to be inserted into the body only after the needle 300 incises the skin.

### Needle and Electrochemical Sensor

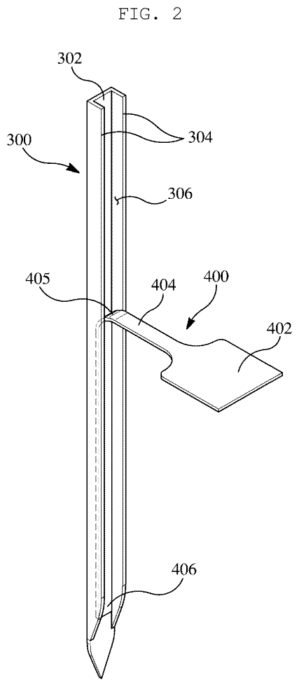

The arrangement relationship between the needle 300 and the electrochemical sensor 400 will be described with reference to FIGS. 2 to 5.

The needle 300 may have an open portion 306 exposing the inside of the needle 300 to the outside and extending along the longitudinal direction of the needle 300. A portion of the distal portion 406 or the bending portion 405 may be attached to or face the needle 300 so as to be located inside the open portion 306 upon invasive insertion into the body.

The distal portion 406 and the proximal portion 402 may lie on different planes having a predetermined angle. A bending direction of the bending portion 405 may coincide with a direction in which the inside of the needle 300 is exposed to the outside by the open portion 306.

A portion where the proximal portion 402 is electrically connected to the transmitter 200 may be located in a direction in which the inside of the needle 300 is exposed to the outside by the open portion 306.

For example, the distal portion 406 may be inserted orthogonally to the skin surface to reduce pain and discomfort. When the main substrate 202 is positioned parallel to a bottom surface of the transmitter 200, the proximal portion 402 may be positioned parallel to the main substrate 202, and the proximal portion 402 may be positioned parallel to the skin surface. In this case, the proximal portion 402 parallel to the skin and the distal portion 406 orthogonal to the skin may lie on different planes orthogonal to each other. The bending portion 405 may be bent along a direction in which the inside of the needle 300 is exposed to the outside.

The needle 300 may include a central wall portion 302 guiding invasive insertion of the electrochemical sensor 400, and opposite sidewall portions 304 preventing the electrochemical sensor 400 from being separated from the needle 300 during invasive insertion.

The central wall portion 302 may prevent the distal portion 406 or the bending portion 405 from protruding in a first axial direction. The first axial direction may refer to a direction in which the inside of the needle 300 is exposed to the outside. When the distal portion 406 or the bending portion 405 protrudes in the first axial direction, the electrochemical sensor 400 may be buckled as a protruding portion thereof is caught on the skin, and only the needle is inserted into the skin but the electrochemical sensor 400 may be come out of the skin.

The sidewall portions 304 may prevent a portion of the distal portion 406 or the bending portion 405 from being separated in a second axial direction. The second axial direction may refer to a direction orthogonal to the first axial direction. The first axial direction, the second axial direction, and the insertion direction may correspond to the axes of an orthogonal coordinate system.

The sidewall portions 304 may be disposed to have a predetermined angle with the center wall portion 302. The predetermined angle may be an angle within a range of 0 to 180 degrees with respect to surfaces of the sidewall portions 304 facing the electrochemical sensor 400.

The inner space of the needle 300 surrounded by the central wall portion 302 and the sidewall portions 304 may communicate with the outside through the open portion 306.

The electrochemical sensor 400 may have a flat plate shape. The electrode 424 of the distal portion 406 may be disposed on one or opposite surfaces of a flat plate portion.

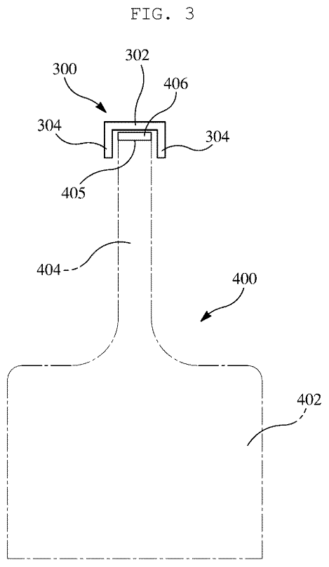

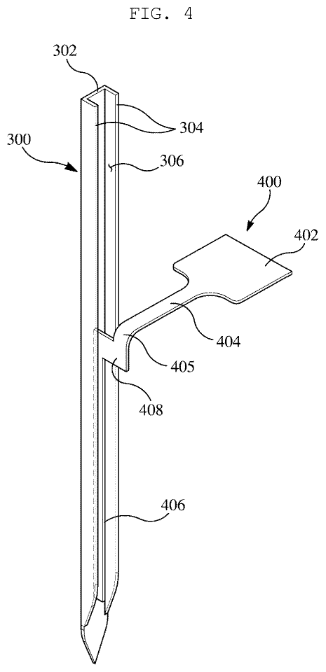

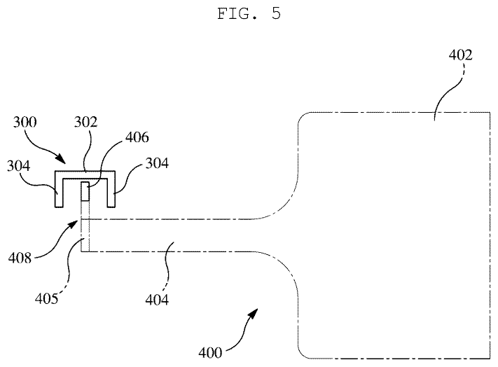

The first embodiment illustrated in FIGS. 2 and 3 may be a case in which the central wall portion 302 faces the electrochemical sensor 400 in parallel. The second embodiment illustrated in FIGS. 4 and 5 may be a case in which the central wall portion 302 faces the electrochemical sensor 400 orthogonally.

In the case of the first embodiment, the electrode 424 may make large-area contact with the outside through the open portion 306. The bending portion 405 may be bent without twisting or changing direction. In the case of the first embodiment, the bending portion 405 may be bent only one time. In this case, a torsional load applied to the bending portion 405 may be low, thereby reducing stress required to maintain a bent state.

In the case of the second embodiment, the electrochemical sensor 400 may be bent while twisting or changing in direction. The intermediate portion 404 may be bent while twisting a plurality of times or changing in direction a plurality of times so that the intermediate portion 404 extends to the proximal portion 402 while avoiding the sidewall portions 304 of the needle 300.

A side extension portion 408 may be formed so that the number of twists of the intermediate portion 404 twisting until reaching the main substrate 202 is reduced and the sidewall portions 304 of the needle 300 being raised is not caught by the intermediate portion 404 or the proximal portion 402 of the electrochemical sensor 400.

The side extension portion 408 may be a portion extending from the intermediate portion 404 in the first axial direction between the distal portion 406 and the proximal portion 402. The intermediate portion 404 adjacent to the distal portion 406 may lie on the same plane as the distal portion 406. Thus, the side extension portion 408 may be a portion extending from the intermediate portion 404 on the same plane as the distal portion 406 in the first axial direction in which the inside of the needle 300 is exposed to the outside.

In the case of the second embodiment, notches may be formed by partially cutting the intermediate portion 404 at positions adjacent to the bending portion 405. This is to minimize twisting or bending of a portion of the intermediate portion 404 with respect to the bending portion 405.

### Electrochemical Sensor

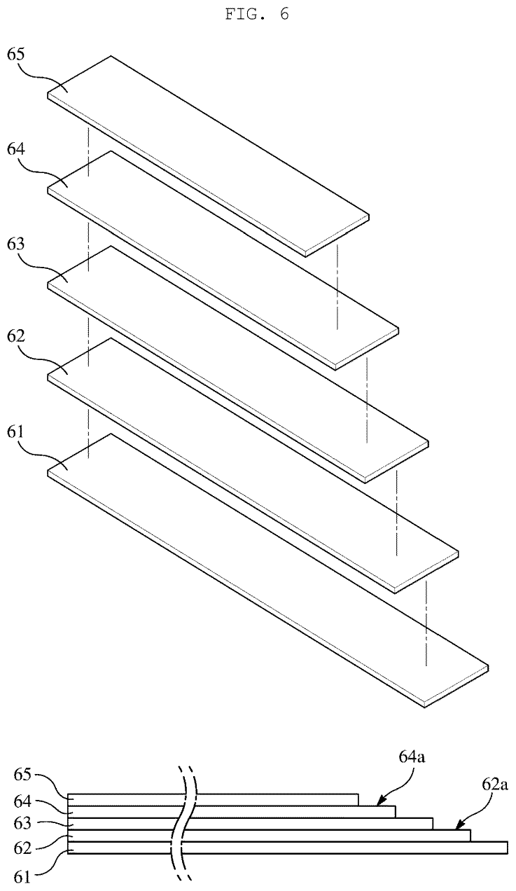

The electrochemical sensor 400 according to the present disclosure will be described with reference to FIGS. 6 to 10.

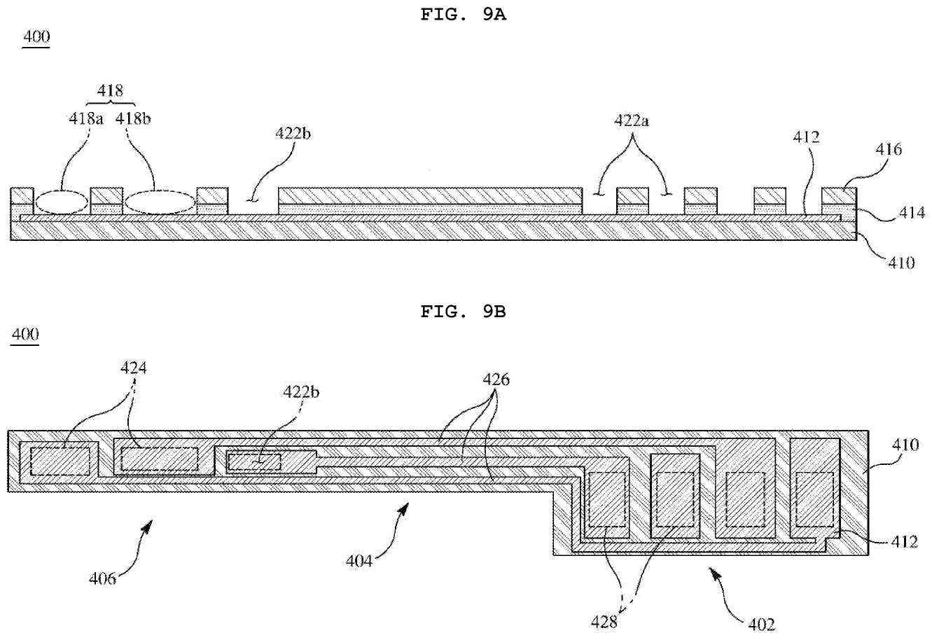

FIGS. 9A and 9B are views illustrating the structure of the electrochemical sensor 400 according to the present disclosure.

FIGS. 9A and 9B illustrate a case where the electrode 424 and the sensor pad 428 are formed on the same one surface of the electrochemical sensor 400.

However, the present disclosure can be applied not only to the case where the electrode 424 and the sensor pad 428 are formed on one surface of the electrochemical sensor 400, but also to the case where the electrode 424 and the sensor pad 428 are formed on opposite surfaces of the electrochemical sensor 400.

The electrochemical sensor 400 according to the present disclosure may selectively react with a part of various analytes including glucose in the body through the electrode 424 of the distal portion 406 invasively inserted into the body.

When a voltage is applied to the electrode 424, an analyte in the body including glucose may be oxidized or reduced, generating electrons, and a current may flow due to flow of the electrons. The generated current may be determined according to the concentration of the analyte in the body, so that a signal of a bio-marker including blood glucose level may be quantified.

A plurality of electrodes 424 inserted into the body and undergoing an oxidation or reduction reaction with glucose may be formed at the distal portion 406. The electrodes 424 may include at least one of a working electrode, a counter electrode, and a reference electrode.

Referring to FIGS. 9A and 9B, a plurality of sensor pads 428 connected to the electrodes 424 may be formed at the proximal portion 402. A current resulting from an electrochemical reaction of the glucose at the distal portion 402 may flow to the sensor pads 428 of the proximal portion 402 along a plurality of leads 426 formed on the base layer 410. The sensor pads 428 may be electrically connected to the main substrate 202.

The electrodes 424 may include at least one or more working electrodes and the reference electrode. The counter electrode may be formed in plural as needed. The counter electrode may be provided when three or more types of electrodes are used for precise data acquisition.

The working electrode may be a porous platinum electrode, and may be manufactured from porous platinum colloid.

The reference electrode may be an electrode that has a constant potential and thus serves as a reference. The reference electrode may be one of a silver chloride (Ag/AgCl) electrode, a calomel electrode, and a mercury sulfate (I) electrode. When the bio-marker is glucose, the silver chloride (Ag/AgCl) electrode may be used as the reference electrode for invasive applications.

In the case of the invasive electrochemical sensor 100, the size thereof needs to be minimized as much as possible for reasons such as pain relief during invasive insertion and discomfort reduction during wearing. As the size of the electrochemical sensor 400 decreases, the area of the electrodes 424 may also decrease. When the area of the electrodes 424 is not sufficiently secured, signal disturbance may occur due to noise. Thus, during manufacture of the electrochemical sensor 400, it is required to consider both aspects of reducing the size of the sensor 100 and securing the area of the electrodes 424.

An insertion length at which the invasive electrochemical sensor 400 is inserted into the skin may range from 3 to 12 mm. When the insertion length is equal to or less than 3 mm, stability of the sensor itself and signal stability may deteriorate due to movement of the body after the sensor is inserted into the body. When the insertion length exceeds 12 mm, the sensor may be located in a range where pain points in the body are distributed, resulting in severe pain and damage to tissues in vivo, such as blood vessels and nerves. In addition, an inserted portion of the distal portion 406 may have a width ranging from 100 to 600 μm. The inserted portion of the distal portion 406 may have a thickness ranging from 10 to 300 μm, preferably from 50 to 150 μm.

Since at least a portion of the distal portion 406 is inserted into the body, when the width of the distal portion 406 is too wide, pain and discomfort may increase during invasive insertion. Thus, it is required to reduce the width of the distal portion 406 to equal to or less than a predetermined width (e.g., 600 μm). When three or more electrodes 424 are all disposed on only one surface of the distal portion 406 invasively inserted into the body, in terms of measurement data, the width of the distal portion 406 needs to be widened to secure a space for the three or more electrodes and leads 426 connected thereto, but in terms of pain relief, the width may be limited to equal to or less than the predetermined width (e.g., 600 μm). Both of such trade-off relationships need to be satisfied.

The electrodes 424 of the distal portion 406 may extend along the base layer 410 via the leads 426 and be electrically connected to the sensor pads 428 of the proximal portion 402. Since the leads 426 are disposed at the intermediate portion 404, when the bending portion 405 is bent, the leads 426 may also be bent.

When the transmitter 200 is attached to the skin and the electrochemical sensor 400 is invasively inserted into the body, the bending portion 405 may remain bent for a considerable period of time. To reduce the torsional load applied to the bending portion 405, the intermediate portion 404 or the bending portion 405 may have a narrower width than the proximal portion 402 or the distal portion 406.

The number of the leads 426 formed at the intermediate portion 404 or the bending portion 405 may be increased in proportion to the number of the electrodes disposed at the distal portion 406. As the number of the leads 426 disposed at the bending portion 405 is increased, insulation may deteriorate and a short circuit may occur. Thus, it is required to optimize the width between the leads 426, the number of the leads 426, the number of the electrodes 424, and the width of the bending portion 405.

A trench 420 may be formed by laser etching a conductive layer 412. The trench 420 formed by laser etching may have a width W1 or W2 ranging from 2 to 200 μm. The width of the trench may be increased as a laser head for laser irradiation is moved a plurality of times and performs laser etching a plurality of times.

The electrodes and the sensor pads may be formed by a laser etching method in which the conductive layer is partially removed by irradiation of a laser beam onto the conductive layer. After the conductive layer is deposited, edge boundaries of the electrodes and edge boundaries of the sensor pads may be formed. The leads respectively connecting the electrodes and the sensor pads to each other may be formed by partially cutting the conductive layer in vertical directions, like the electrodes and the sensor pads. An insulating layer may be attached after the edge boundaries of the electrodes and the edge boundaries of the sensor pads are formed.

The trench may be etched into the conductive layer, thereby forming conductive islands. The trench may have a height equal to the thickness of the conductive layer. The conductive layer, the electrodes, and the sensor pads may all have the same thickness.

The electrochemical sensor may have a width equal to or less than 600 μm, and a length equal to or less than 3 cm. The electrodes and the sensor pads may have a width equal to or less than 500 μm, and the leads may have a width equal to or less than 150 μm. At least two electrodes and at least two leads may be formed on one surface of the distal portion of the electrochemical sensor.

The electrodes and the trench may be formed without burrs by laser etching no matter how complicated the pattern of the electrodes is and how narrow the width of the trench is. For process simplification, the conductive layer is preferably formed by sputtering a metal over the entire exposed area of the base layer. When forming double-side electrodes, the metal may be sputtered on both top and bottom surfaces of the base layer in which a via hole is formed.

The electrodes 424 or the leads 426 may be electrically separated from each other by the trench 420. As the width of the trench 420 becomes narrower, a sufficient area of the electrodes 424 for reacting with the analyte may be secured. On the contrary, as the width of the trench 420 becomes narrower, insulation may deteriorate. Laser etching for trench formation may satisfy trade-off relationships between miniaturization and insulation. As the width of the bending portion 405 becomes narrower, a torsional force may be reduced, and fatigue failure may be prevented even when the bending portion 405 remains bent and fixed for a considerable period of time.

By the trench 420, it is easy to secure a sufficient area for the leads 426, the electrodes 424, or the sensor pads 428, thereby improving the signal transmission rate and reducing the short-circuit defect rate.

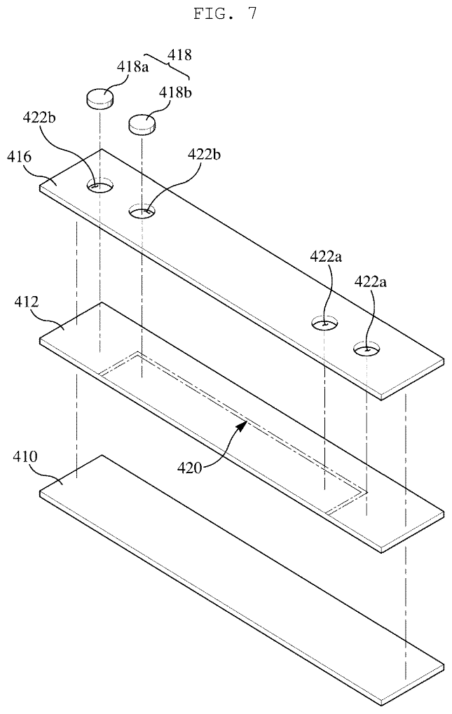

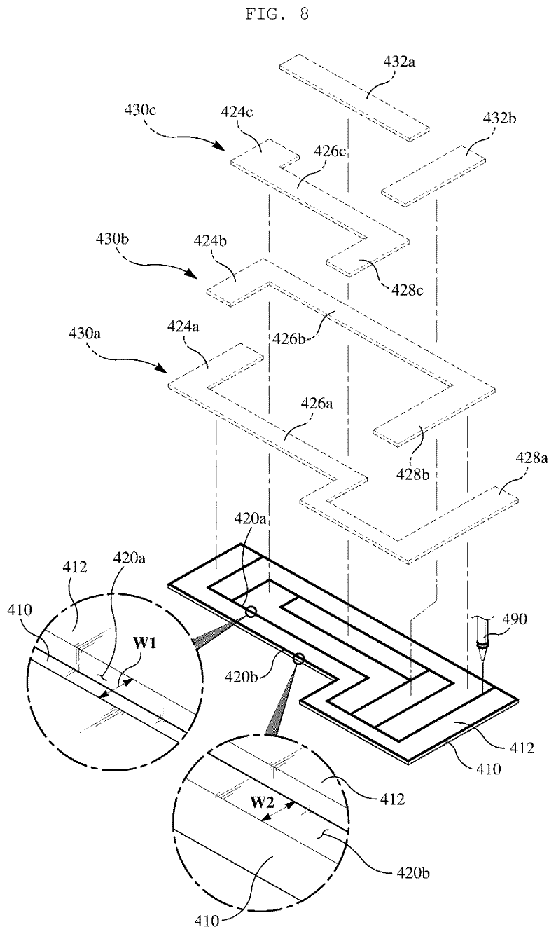

FIG. 6 is a view illustrating a comparative embodiment of the present disclosure, and can be compared with FIGS. 7 and 8, which illustrates a method of manufacturing the electrochemical sensor 400 according to the present disclosure. FIG. 6 illustrates a comparative embodiment for forming two electrodes of a sensor, a first electrode 62a and a second electrode 64a.

In the comparative embodiment, a first electrode layer 62, a first insulating layer 63, a second electrode layer 64, and a second insulating layer 65 may be sequentially stacked on a base layer 61 constituting a body of the sensor 100. To form the two electrodes, a portion of each layer for electrode formation may sequentially increase in length in the order of the base layer 61, the first electrode layer 62, the first insulating layer 63, the second electrode layer 64, and the second insulating layer 65. Due to such a difference in length of each layer, the first electrode 62a may be exposed on the first electrode layer 62 and the second electrode 64a may be exposed on the second electrode layer 64.

In the comparative embodiment, as the number of the electrodes is increased, the number of the electrode layers and insulating layers is increased, the thickness of the sensor is increased, and pain and discomfort may be increased upon insertion of the sensor into the skin. When the number of the electrodes is increased to improve reactivity with the analyte in the body, the thickness of a distal portion of the sensor may become too thick. Even when the width of each stacked layer is narrowed, the number of the stacked layers according to the increase in the number of the electrodes cannot be fundamentally reduced, and as a result, it may be difficult to minimize the thickness of the sensor that is invasively inserted into the body.

Referring to FIG. 7, the electrochemical sensor 400 may include the base layer 410 that is flexible so as to be bendable upon invasive insertion of the sensor into the body. The base layer 410 may include at least one of synthetic resin, polyimide (PI), and polyethylene terephthalate (PET) as an insulating material. The base layer or the insulating layer may have a thickness equal to or less than 100 μm.

The conductive layer 412 may be formed on the base layer 410 by sputtering or the like. The conductive layer deposited by ejecting metal atoms or molecules may have a thickness equal to or less than 10 μm. The conductive layer may be formed by sputtering the metal over the entire exposed area of the base layer before the edge boundaries of the electrodes and the edge boundaries of the sensor pads are formed.

To satisfy trade-off relationships between miniaturization and insulation, the electrodes and the sensor pads may be formed by a laser etching method in which the conductive layer is partially removed by irradiation of a laser beam onto the conductive layer.

The trench 420 may be formed in the conductive layer 412 prior to bonding the insulating layer 416 to the conductive layer 412. The conductive layer 412 may be divided into different members by the trench 420. By the trench 420, the conductive layer 412 may be divided into different electrodes 424, divided into different leads 426, and divided into different sensor pads 428.

After forming the conductive layer 412, the insulating layer 416 may be attached. The insulating layer may be adhered onto the conductive layer in a state in which portions of the insulating layer corresponding to the electrodes and the sensor pads are removed so that the electrodes and the sensor pads are exposed to the outside.

The portions of the insulating layer 416 may be removed by a cutter or a puncher. When the size of an opening of the insulating layer is small and micromachining is required, the laser etching method used for forming the trench in the conductive layer may be used for forming the opening in the insulating layer.

The laser etching method may also be applied to the base layer. Since a via hole required for formation of double-side electrodes requires micromachining, the laser etching method used to form the trench of the conductive layer may be used for forming the via hole in the base layer. The via hole may be formed by partially cutting the base layer. The conductive layer may be sputtered with the same metal material continuously seamlessly along a top surface of the base layer, a surface of the via hole, and a bottom surface of the base layer.

A plurality of openings 422 may be formed through the insulating layer 416. The electrodes and the sensor pads formed on the conductive layer may be exposed to the outside through the openings. A proximal opening 422a may be formed at the proximal portion 402 and a distal opening 422b may be formed at the distal portion 406. A portion of each of the sensor pads 428 may be exposed to the outside through the proximal opening 422a. The portion of each of the sensor pads 428 exposed through the proximal opening 422a may be electrically connected to a contact pad of the main substrate 202.

A portion of each of the electrodes 424 may be exposed to the outside through the distal opening 164. The portion of each of the electrodes 424 exposed through the distal opening 164 may make contact with the interstitial fluid or bloodstream and cause an electrochemical reaction with the analyte.

The electrochemical sensor 400 according to the present disclosure may include a porous selective transmission layer 418 surrounding surfaces of the electrodes 424. The selective transmission layer 418 may be applied to the electrodes 424 of the distal portion 406 for reacting with the analyte in the body.

The selective transmission layer 418 may have a mesoporous structure having pores with a size ranging from 2 to 50 nm.

The material of the selective transmission layer 418 may be determined according to the type of the analyte to be reacted with the electrodes 424 and may vary according to the type of the electrodes 424 to which the selective transmission layer 418 is applied. For example, when the analyte is glucose and the electrodes 424 to which the selective transmission layer 418 is applied is working electrodes, the material of the selective transmission layer 418 may be mesoporous platinum. Porous platinum may be fabricated from porous platinum colloids. When the analyte is glucose and the electrodes 424 to which the selective transmission layer 418 is applied are reference electrodes, the material of the selective transmission layer 418 may be silver chloride (Ag/AgCl).

The selective transmission layer 418 may be applied to the electrodes 424 through the respective distal openings 422b in a state in which the base layer 410, the conductive layer 412, and the insulating layer 416 are stacked. When a plurality of distal openings 422b face different types of electrodes, a first selective transmission layer 418a and a second selective transmission layer 418b may include different types of materials.

FIG. 8 is a view illustrating in detail the trench 420 of the conductive layer 412. FIG. 8 schematically illustrates the entire electrochemical sensor 400 from the proximal portion 402 to the distal portion 406.

Referring to FIG. 8, after the conductive layer 412 is deposited on the base layer 410 by sputtering or the like, the trench 420 may be formed by laser etching or the like.

A plurality of conductive islands separated from each other may be provided on the conductive layer 412 by laser etching or the like. The conductive islands may each form a closed surface and be electrically insulated from each other.

The base layer 410 may be exposed under the trench 420, and adjacent conductive islands 430 may be insulated from each other by the trench 420.

Each of the sensor pads 428 may be formed on each of the conductive islands 430 at a position corresponding to the proximal portion 402, each of the leads 426 may be formed on each of the conductive islands 430 at a position corresponding to the intermediate portion 404 or the bending portion 405, and each of the electrodes 424 may be formed on each of the conductive islands 430 at a position corresponding to the distal portion 406.

The conductive islands may include a conductive island in which portions thereof corresponding to each of the electrodes and each of the sensor pads are exposed to the outside through cut portions of the insulating layer, and a dummy portion entirely covered with the insulating layer so as not to be exposed to the outside.

A first conductive island 430a, a second conductive island 430b, and a third conductive island 430c including different electrodes 424 may be formed. The first conductive island 430a may include a first sensor pad 428a at a position corresponding to the proximal portion 402, a first lead 426a at a position corresponding to the bending portion 405, and a first electrode 424a at a position corresponding to the distal portion 406.

The second conductive island 430b may include a second sensor pad 428b at a position corresponding to the proximal portion 402, a second lead 426b at a position corresponding to the bending portion 405, and a second electrode 424b at a position corresponding to the distal portion 406. The third conductive island 430c may include a third sensor pad 428c at a position corresponding to the proximal portion 402, a third lead 426c at a position corresponding to the bending portion 405, and a third electrode 424c at a position corresponding to the distal portion 406.

Each of the first electrode 424a, the second electrode 424b, and the third electrode 424c may be any one of the working electrode, the counter electrode, and the reference electrode.

When forming the conductive islands 430 that are separated from each other while forming closed surfaces, the dummy portion 432 may be formed between the conductive islands 430. The dummy portion 432 may be used as a conductive island 430 having an electrode 424 or a sensor pad 428 when the insulating layer is exposed. The dummy portion 432 may be completely removed by repeated laser etching or the like. However, removal of the dummy portion 432 is not necessary because only the electrical isolation by the trench needs to be achieved. This is another advantage of the present disclosure.

When the dummy portion 432 is formed too wide in a portion of the conductive layer 412 covered with the insulating layer 416 thereon after the trench 420 is patterned, the dummy portion 432 may remain without being removed in order to prevent a portion of the insulating layer 416 from collapsing down.

The trench 420 may include an electrode trench 420a or an edge trench 420b. The electrode trench 420a may insulate between the conductive islands 430. The electrode trench 420a may be disposed at least one of between the electrodes 424, between the leads 426, and between the sensor pads 428.

Meanwhile, when the conductive layer is exposed to the edge of the electrochemical sensor, insulation may deteriorate. Thus, it is required to prevent side surfaces of the conductive layer from being exposed. After the conductive layer is deposited, the conductive layer may be partially cut along the edge of the electrochemical sensor. As a result, the edge trench 420b may be formed. Thus, the insulating layer may be attached onto the base layer at the edge of the electrochemical sensor, thereby forming insulation. The insulating layer may be attached onto the conductive layer stacked on the base layer at a position inside the edge of the electrochemical sensor.

The edge trench 420b may form an outermost edge of the conductive layer 412. The edge trench 420b may serve to insulate a conductive island 430 located at the outermost side of the electrochemical sensor 400 from the outside of the sensor 400. When the electrochemical sensor 400 is processed as an array, the edge trench 420b may separate adjacent sensors 400 or adjacent conductive islands 430 from each other, thereby preventing a short circuit from occurring therebetween.

The width W1 of the electrode trench 420a and the width W2 of the edge trench 420b may range from 5 to 30 μm.

A bonding layer 414 for attaching the insulating layer 416 to the conductive layer 412 may be provided. The bonding layer 414 may be positioned between the conductive layer 412 and the insulating layer 416. When the openings 422 are formed in the insulating layer 416, the openings 422 may also be formed in the bonding layer 414.

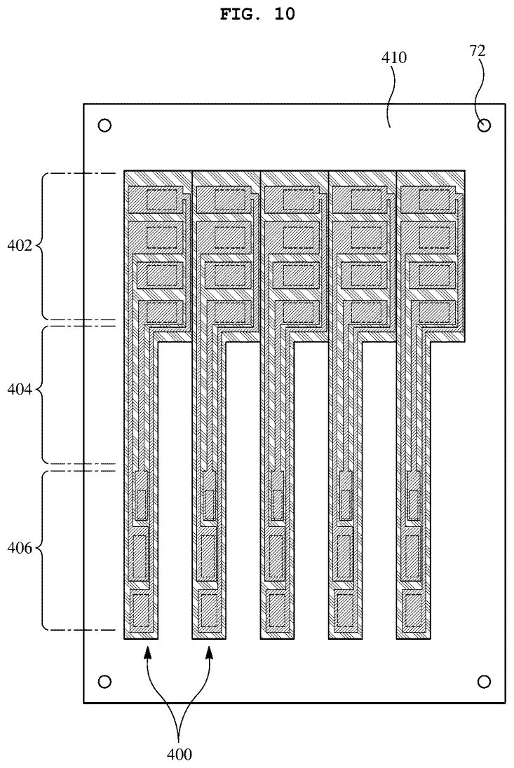

FIG. 10 is a view illustrating a sensor array for manufacturing a plurality of electrochemical sensors 400 simultaneously. In order to repeatedly form the selective transmission layer, at least one of dip coating, spray coating, and pasting may be performed.

Referring to FIG. 10, alignment holes 72 may be formed in the base layer 410. Alignment pins of a jig may be inserted into the alignment holes 72. As the alignment holes 72 of the base layer 410 and alignment holes (not illustrated) of the insulating layer 416 are aligned with each other, each opening of the insulating layer 416 may be aligned with an electrode or a sensor pad.

The plurality of electrochemical sensors 400 may be simultaneously manufactured in the form of an array in which the sensors are connected to each other, and then may be separated from each other individually.

The electrochemical sensors 400 may form a sensor array by connecting respective base layers 410 thereof to each other. The electrochemical sensors 400 may be formed by performing at least one of forming the conductive layer 412 and forming the trench 420 by laser etching simultaneously for each sensor on one base layer 410. Forming the insulating layer 416 and forming the selective transmission layer 418 may also be performed simultaneously for each sensor.

What is claimed is:

1. A continuous analyte meter comprising:
an electrochemical sensor including a distal portion having a plurality of electrodes reacting with an analyte in the body and a proximal portion having a plurality of sensor pads connected to the electrodes; and
a transmitter attached to the skin, the transmitter including a main substrate on which at least one of a power supply unit, a communication unit, and a control unit is formed, and a housing in which the main substrate is accommodated,
wherein the distal portion of the electrochemical sensor is disposed at an exposed portion of a needle exposed along a longitudinal direction of the needle,
the distal portion of the electrochemical sensor is inserted into the body after the skin is incised by the needle,
the electrochemical sensor has flexibility that it is impossible to penetrate the skin alone without the needle, and
the electrochemical sensor comprises a flexible base layer, a conductive layer stacked on the base layer, and an insulating layer attached on the conductive layer.

2. The continuous analyte meter of claim 1, wherein the electrochemical sensor has a thickness equal to or less than 300 μm.

3. The continuous analyte meter of claim 1, wherein the base layer or the insulating layer has a thickness equal to or less than 100 μm, and
the conductive layer has a thickness equal to or less than 10 μm.

4. The continuous analyte meter of claim 1, wherein a plurality of leads respectively connecting the electrodes and the sensor pads to each other are formed by partially cutting the conductive layer in vertical directions, like the electrodes and the sensor pads.

5. The continuous analyte meter of claim 1, wherein a plurality of leads respectively connecting the electrodes and
the sensor pads to each other are formed on the conductive layer, the electrochemical sensor has a width equal to or less
than 600 μm, and a length equal to or less than 3 cm, the electrodes and the sensor pads have a width equal to or less than are 500 μm,
the leads have a width equal to or less than 150 μm, and
at least two electrodes and at least two leads are formed on one surface of the distal portion of the electrochemical sensor.

6. The continuous analyte meter of claim 1, wherein the conductive layer is formed by sputtering a metal on the base layer.

7. The continuous analyte meter of claim 1, wherein the conductive layer is formed by sputtering a metal over the entire exposed area of the base layer before edge boundaries of the electrodes and edge boundaries of the sensor pads are formed.

8. The continuous analyte meter of claim 1, wherein the conductive layer is formed by sputtering a metal over the entire exposed area of the base layer before edge boundaries of the electrodes and edge boundaries of the sensor pads are formed,
the edge boundaries of the electrodes and the edge boundaries of the sensor pads are formed after the conductive layer is deposited,
an insulating layer is attached after the edge boundaries of the electrodes and the edge boundaries of the sensor pads are formed, and
the conductive layer, the electrodes, and the sensor pads all have the same thickness.

9. The continuous analyte meter of claim 1, wherein the conductive layer is formed by sputtering a metal over the entire exposed area of the base layer,
the conductive layer is partially cut along an edge of the electrochemical sensor after the conductive layer is deposited,
the insulating layer is attached onto the base layer at the edge of the electrochemical sensor, and
the insulating layer is attached onto the conductive layer stacked on the base layer at a position inside the edge of the electrochemical sensor.

10. The continuous analyte meter of claim 1, wherein the electrodes and the sensor pads are formed by a laser etching method in which the conductive layer is partially removed by irradiation of a laser beam onto the conductive layer.

11. The continuous analyte meter of claim 1, wherein the insulating layer is adhered onto the conductive layer in a state in which portions of the insulating layer corresponding to the electrodes and the sensor pads are removed so that the electrodes and the sensor pads are exposed to the outside.

12. The continuous analyte meter of claim 1, wherein a via hole is formed by partially cutting a base layer, and
the conductive layer is sputtered with the same metal material continuously seamlessly along a top surface of the base layer, a surface of the via hole, and a bottom surface of the base layer.

13. The continuous analyte meter of claim 1, wherein a plurality of conductive islands separated from each other are provided on the conductive layer by laser etching for partially removing the conductive layer with a laser beam irradiated onto the conductive layer, and
each of the conductive islands forms a closed surface.

14. The continuous analyte meter of claim 1, wherein a trench is formed in the conductive layer by laser etching for partially removing the conductive layer with a laser beam irradiated onto the conductive layer, and
the trench is etched into the conductive layer.

15. The continuous analyte meter of claim 1, wherein a trench is formed in the conductive layer by laser etching for partially removing the conductive layer with a laser beam irradiated onto the conductive layer,
the trench has a width ranging from 2 to 200 μm, and
the trench has a height equal to a thickness of the conductive layer.

16. The continuous analyte meter of claim 1, wherein a trench is formed in the conductive layer by laser etching for partially removing the conductive layer with a laser beam irradiated onto the conductive layer, and
a width of the trench is increased as a laser head for laser irradiation is moved a plurality of times and performs laser etching a plurality of times.

17. The continuous analyte meter of claim 1, wherein a plurality of conductive islands separated from each other are provided on the conductive layer by laser etching for partially removing the conductive layer with a laser beam irradiated onto the conductive layer, and
the conductive islands comprise a conductive island in which portions thereof corresponding to each of the electrodes and each of the sensor pads are exposed to the outside through cut portions of the insulating layer, and a dummy portion entirely covered with the insulating layer so as not to be exposed to the outside.

18. The continuous analyte meter of claim 1, wherein the electrochemical sensor further comprises a bending portion that connects the proximal portion and the distal portion to each other, is disposed between the proximal portion and the distal portion, and is flexibly bendable,
a plurality of conductive islands separated from each other are formed on the conductive layer,
a current resulting from an electrochemical reaction of glucose at the distal portion flows to the sensor pads of the proximal portion along a plurality of leads formed on the base layer, and
each of the sensor pads is disposed on each of the conductive islands at a position corresponding to the proximal portion, each of the leads is disposed on each of the conductive islands at a position corresponding to the bending portion, and each of the electrodes is disposed on each of the conductive islands at a position corresponding to the distal portion.

19. The continuous analyte meter of claim 1, wherein a plurality of openings are formed through the insulating layer,
the openings comprise a proximal opening exposing each of the sensor pads to the outside and a distal opening exposing each of the electrodes to the outside,
a selective transmission layer is applied to the openings,
a material of the selective transmission layer is determined according to a type of the analyte to be electrochemically reacted with the electrodes,
when the electrodes are reference electrodes, the selective transmission layer includes Ag/AgCl, and
when the electrodes are working electrodes, the selective transmission layer has a mesoporous structure and the selective transmission layer includes platinum.

20. The continuous analyte meter of claim 1, wherein a plurality of electrochemical sensors are simultaneously manufactured in the form of an array in which the sensors are repeatedly arranged, and then are separated from each other individually.
